(19) United States
(12) Patent Application Publication
(10) Pub. No.: US 2021/0186801 A1
(43) Pub. Date: Jun. 24, 2021

(54) PERSONALIZED DEVICE TO AUTOMATICALLY DETECT AND REDUCE MUSCLE SPASMS WITH VIBRATION

(71) Applicant: UNIVERSITY OF MIAMI, Miami, FL (US)

(72) Inventors: Christine Thomas, Miami, FL (US); Jorge Bohorquez, Doral, FL (US); Khema Sharma, Miami, FL (US); Bradley Deforest, Pembroke Pines, FL (US)

(21) Appl. No.: 17/273,494

(22) PCT Filed: Sep. 5, 2019
(86) PCT No.: PCT/US19/49766

§ 371 Date: Mar. 4, 2021

Related U.S. Application Data
(60) Provisional application No. 62/727,324, filed on Sep. 5, 2018.

(51) Int. Cl. A61H 23/02

(52) U.S. Cl. CPC A61H 23/02; A61H 2201/0157; A61H 2230/605; A61H 2201/5007; A61H 2205/10; A61H 2201/165

(57) ABSTRACT

A personalized wearable device can detect muscle contractions, such as spasms, and provide vibrational energy that treats these muscle contractions.

### CROSS-REFERENCE TO RELATED APPLICATIONS

This application claims the priority benefit under 35 U.S.C. § 119(e) of U.S. Provisional Patent Application No. 62/727,324, filed Sep. 5, 2018, the disclosure of which is incorporated herein by reference in its entirety.

### STATEMENT OF GOVERNMENT SUPPORT

This invention was made with government support under 1R01NS100810-01A1 awarded by the National Institute of Health. The government has certain rights in the invention.”

### FIELD OF THE INVENTION

The present disclosure relates to detection of muscle contractions (spasms, etc.) and, more particularly, to a personalized device for detecting muscle contractions and responding with a vibratory treatment to reduce the same.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Spasticity is a debilitating health problem for individuals with neurological disorders, including, in particular, individuals that have suffered strokes, spinal cord injuries (SCI), and the like. For these individuals, involuntary muscle activity (also termed “spasms”) can occur throughout the day and night. At the most severe, these spasms interfere with bodily functions (e.g., bladder function), exacerbate pain, disrupt activities (e.g., sleep), and lower the overall quality of life for the individual. The current gold standard treatment for spasms is oral baclofen. However, as a treatment, it is often ineffective, and intolerance and side effects such as drowsiness are common.

Thus, there is a need for an effective spasticity treatment for individuals with neurological disorders that will improve their quality of life.

### SUMMARY OF THE INVENTION

The present application provides for a personalized wearable device that can detect muscle contractions, such as spasms, and provide vibrational energy that treats these muscle contractions.

Involuntary muscle activity (spasms) is the most debilitating aspect of spasticity after spinal cord injury (SCI) because the contractions interfere with everyday tasks, and limit rehabilitation. Treatments are not always effective, lowering health-related quality of life.

In some examples, the present techniques provide a closed-loop control of muscle or tendon vibration to implement clinically meaningful management of muscle contractions.

In some examples, the present techniques include a wearable device having housing configured to deliver vibration to target regions of a subject.

In some examples, the present techniques include methods to determine the vibration parameters that will reduce muscle contractions. For example, the present techniques include methods to determine the vibration parameters (e.g., vibratory stimulation signals that reduce spasms in leg muscles, paralysed by spinal cord injuries or other causes. The present techniques are able to detect such muscle contractions as spasms, whether a subject is in a seated or reclined position, by detecting electromyography (EMG) signals. These muscle contractions can then be treated by applying to the Achilles tendon, muscles, or other target areas, vibratory energy at different frequencies, durations and/or amplitudes to dampen the spasms in real-time using closed-loop control.

In some examples, the present techniques further include assessing the efficacy of tendon and/or muscle vibration in altering muscle spasms by treating spasms as they occur, which personalizes the intervention for maximal clinical and user impact.

In accordance with an example, a system to treat muscle contraction of a subject comprises: one or more processors; one or more memories; a sensor stage having one or more electrodes configured to receive electromyography (EMG) signals from a target muscle of the subject; a muscle contraction analysis stage configured to execute instructions stored on the one or more memories, wherein the instructions when executed cause the one or more processors to, sample the EMG signals in real-time and calculate a plurality of features from the EMG signals, determine whether a predetermined pattern of signal features indicate muscle contraction in the target muscle, and if the pattern of signal features determine the presence of muscle contraction in the target muscle, determine the need for a vibratory stimulus signal to the muscle contraction; and a vibratory motor stage configured to apply the vibratory stimulus signal to the subject to treat the muscle contraction; wherein the sensor stage, the vibratory motor stage, and the muscle contraction analysis stage are in a closed-loop configuration, wherein the sensor stage is configured to receive additional EMG signals, the muscle contraction analysis stage is configured to sample the additional EMG signals in real-time and the vibratory motor stage is configured to stop application of the vibratory stimulus.

In accordance with another example, a computer-implemented method to treat muscle contraction of a subject, the method comprises: receiving, at a processor, electromyography (EMG) signals of the subject, the EMG signals being obtained from a target muscle of the subject; sampling, at the processor, the EMG signals in real-time and calculating a plurality of features from the EMG signals; determining whether a predetermined pattern of signal features indicate contraction in the target muscle; if the pattern of signal features indicate the presence of muscle contraction in the target muscle and determining a vibratory stimulus signal corresponding to the muscle contraction and applying the vibratory stimulus signal to the subject to treat the muscle contraction; and applying the vibratory stimulus signal in a closed-loop configuration, wherein the method further comprises receiving additional EMG signals and sampling the additional EMG signals in real-time, calculating a plurality of features of the additional EMG signal values, and determining when the plurality of feature patterns of the additional EMG signal values indicate muscle contraction.

### BRIEF DESCRIPTION OF THE DRAWINGS

The figures described below depict various aspects of the system and methods disclosed herein. It should be understood that each figure depicts an example of aspects of the present systems and methods.

FIG. 1 is a schematic illustration of an example muscle contraction response system for detecting and treating muscle contraction in a subject, according to an example.

FIG. 2 is flow diagram of a process for detecting and treating muscle contraction in a subject as may be performed by the system of FIG. 1, according to an example.

FIG. 3 is flow diagram of an example implementation of the process of FIG. 2.

### DETAILED DESCRIPTION

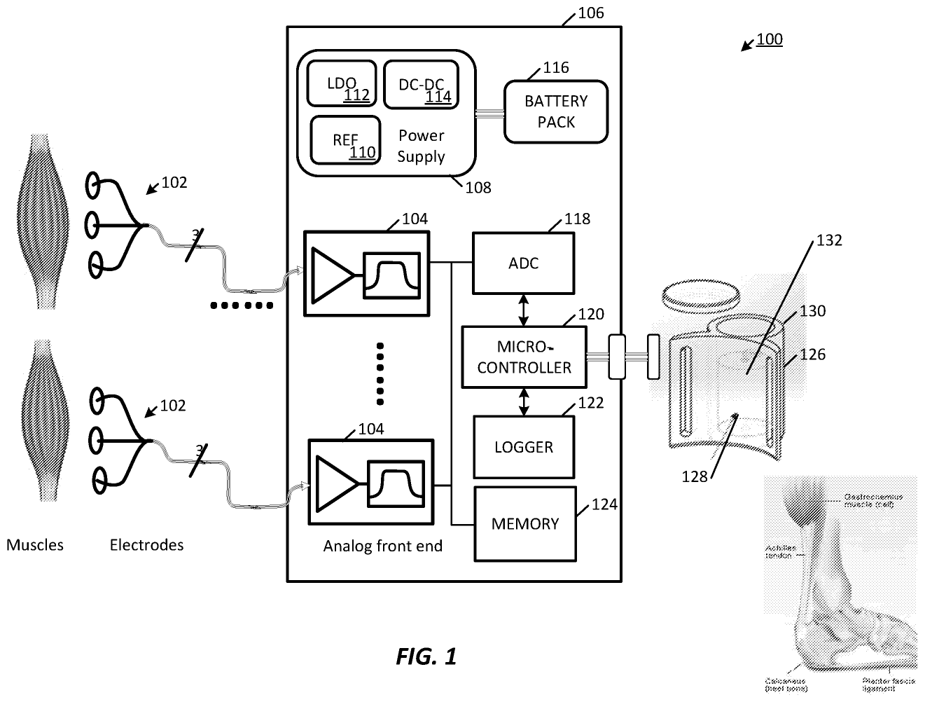

FIG. 1 illustrates a muscle contraction response system 100 having various components used in implementing techniques described herein. Muscle electrodes 102 are provided that may be attached to muscles of a subject matter. The electrodes 102 are sensing electrodes and are configured to sense electrical signal data corresponding to episodes of muscle contraction, including muscle spasms. For example, the electrodes 102 may be electromyography (EMG) electrodes, surface electrodes or intramuscular (sub-surface) electrodes, that record muscle activity above, on, or below the skin of a subject. Any number of electrodes may be used, but in the illustrated example 3 EMG electrodes 102 are used and positioned per muscle, with two different muscle groups shown with electrodes. If muscle activity is to be measured over multiple different muscle (or muscle groups), then multiple groupings of EMG electrodes may be used, as shown in FIG. 1.

Each grouping of EMG electrodes 102 is coupled to a dedicated analog front end circuit 104 that can provide pre-processing such as single amplification and bandpass filtering, to increase the signal to noise ratio in the collected EMG signals. The analog front end circuits are contained within a muscle contraction analysis stage (controller) 106 that additionally includes a power supply 108 configured to provide a reference voltage 110, a low dropout voltage regulator (LDO) 112, and a DC-to-DC converter 114, accessing a battery powering stage 116, that may also be part of the power supply 108. The pre-processed outputs from the analog front end stages are provided to an analog-to-digital converter 118 to produce a digital output signal indicating muscle activity. That signal is provided to a microcontroller 120 (having one or more processors) that is able to store muscle activity data in a logger 122 and that is able to access instructions stored on one or more memories 124, where those instructions, when executed cause the microcontroller to perform processes as described herein.

The muscle contraction analysis stage 106 is communicatively coupled to a vibratory motor stage 126 that engages the subject to provide vibratory stimulation to the subject for treating unwanted muscle activity. The sensing electrodes 102, the vibratory motor stage 126, and the muscle contraction analysis stage 106 may be in a closed-loop configuration, wherein the electrodes 102 are configured to receive additional EMG signals, the muscle contraction analysis stage 106 is configured to sample the additional EMG signals in real-time, and the vibratory motor stage 126 is configured to stop application of the vibratory stimulus to treat the subject. The vibratory motor stage 126 may be mounted to the subject at the location of a target muscle or target region to treat. Example target regions include tendons, such as the Achilles tendon, as shown in the illustrated example, as well as various muscles.

The vibratory motor stage 126 includes an inner engagement surface 128 having a curved shaped for engaging a muscle or tendon of a subject. For example, the inner engagement surface 128 may be shaped and dimensioned to engage the outer skin around the Achilles tendon of a subject to apply vibratory signals to reduce muscle spasms in the muscles of that limb. The vibratory motor stage 126 includes a motor housing 130 within which is mounted a vibratory motor 132. The housing 130 is adjacent to the inner engagement surface and is mechanically coupled to that surface such that vibratory motion of the motor 132 is transferred to the surface for transfer to the subject. The motor 132 is electrically coupled to the muscle contraction analysis stage 106 which controls the timing, amplitude, and frequency of the vibratory output from the motor and does so in response to detection and analysis of muscle contraction.

The muscle contraction analysis stage controller 106 and the sensor electrodes 102 may be part of the vibratory motor stage 126, in some examples. For example, the vibratory motor stage 126 may be a portable wearable device, having the housing 130 formed as a wearable bracelet. The electrodes 102 may be exposed at the inner surface 128 for engagement with a target muscle for the subject, such as a tendon of the subject. The controller 106 may be housed within the housing 130 and communicatively coupled to these electrodes 102 and the motor 132 for providing the closed-loop real-time EMG feature monitoring and analysis and vibratory stimulus signal determination and application to the target muscle at the engagement inner surface 128. In some implementations, sensing electrodes are placed at a different location than the engagement surface for the motor, for example, using a closed bracelet that fits entirely around an ankle or arm, or a bracket type structure that fits on opposing sides of shoulder region of a subject. The wearable structure may include a first engagement surface, such as for an extensor surface, for measuring EMG signals, and a different engagement surface, such as for an flexor surface, for applying the vibratory stimulus signal treatment, or vice versa. In some examples, the sensing electrodes are extendible from the vibratory motor stage 126 for attachment at any of a plurality of different surfaces. In some examples, multiple different sensing electrodes, either extendible from the housing or positioned at different engagement surface locations of the housing, may be provided for sensing EMG signals at multiple different target regions for a subject. Further still, multiple different vibratory motors may be housed within the motor stage for applying vibratory stimulus signals to different target regions, including applying stimulus signals having different vibration parameters at the different regions.

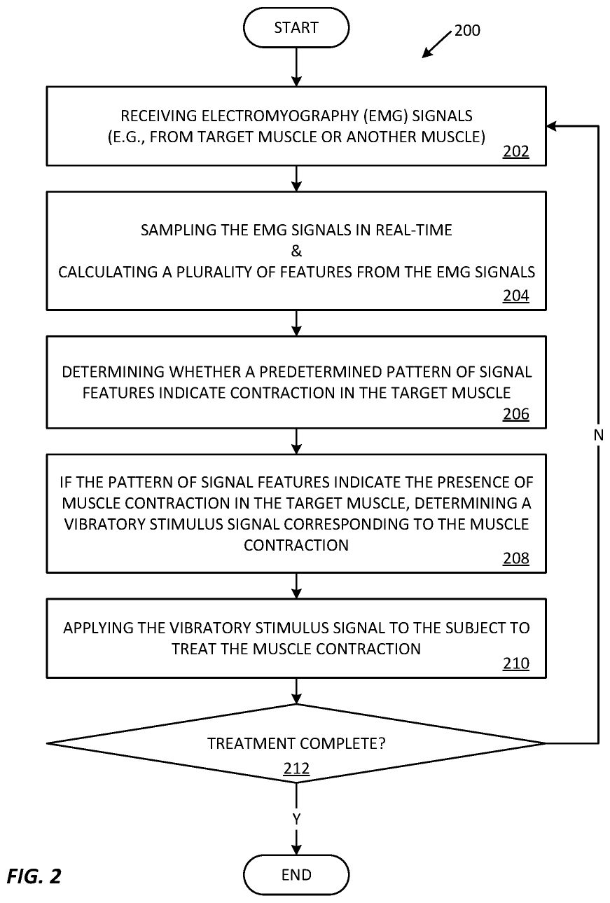

FIG. 2 illustrates a process 200 for detecting and treating muscle contractions as may be implemented by the system of FIG. 1. The process, which may be a computer-implemented method to treat muscle contraction of a subject, includes the following processes. Initially, at a process 202, a microcontroller receives electromyography (EMG) signals from the muscle-engaged electrodes, as collected by the front end analog circuits. At a process 204, the microcontroller may sample the received EMG signals in real-time. The microcontroller calculates a plurality of features from the EMG signals (e.g., amplitude, energy). The features of the EMG may be determined in real time. Example features include the rectified EMG, the EMG moving average, and the time interval for which the rectified EMG moving average is higher than a pre-defined threshold.

At a process 206, the microcontroller may determine whether a predetermined pattern of signal features indicate contraction in a target muscle. For example, a muscle contraction may be identified when the time interval for which the rectified EMG moving average is above a threshold for more than 50 milliseconds. Different predetermined patterns may exist for each different EMG feature identified at process 204. Further the EMG features and the predetermined patterns may be assessed for each different set of electrodes, thereby allowing for analysis at different locations on the body of a subject. If the pattern of signal features indicate the presence of muscle contraction in the target muscle, the microcontroller may determine a vibratory stimulus signal corresponding to the muscle contraction, through a process 208. The microcontroller then sends signals to the vibratory motor stage, at a process 210, which then applies the vibratory stimulus signal to the subject to treat the muscle contraction. A process 212 may then determine if the treatment is complete by analyzing further EMG signals for measured on the subject and determining if the EMG features indicate the continued presence of contraction. The vibratory stimulus signal may be applied in a closed-loop configuration, such that the microcontroller may receive additional EMG signals and sample those additional EMG signals in real-time, calculating a plurality of features of the additional EMG signal values, and determining when the plurality of feature patterns of the additional EMG signal values indicate muscle contraction.

The process 208, for example, may determine vibratory parameters for the vibratory stimulus signal, such as the frequency, duration, and/or amplitudes of the vibratory stimulus signal. In some examples, the frequency range of the vibratory stimulus signal is selected from and including 20 Hz to 120 Hz. This range allows for frequencies to be chosen to stimulate Meissner's corpuscles, muscle spindles, and Pacinian corpuscles. In some examples, the process 208 may determine vibration stimulus signal duration range from and including 0.2 seconds to 1 second, for example, after the muscle contraction is detected. In some examples, the process 208 determines a vibration stimulus signal amplitude range from and including 0.25 millimeters to 2 millimeters. The direction of that stimulus signal will be parallel or transverse to the a treatment are, for example, parallel or transverse to a tendon.

The process 208 may implement an executable protocol to find the vibration parameters for a specific subject for treating the muscle contraction. In an example, the process 208 accesses a stored database of vibration parameters tested and determined to correspond to treatments for previously measured EMG signal features. In another example, vibrations parameters are determined for a specific subject by using a testing protocol that cycles through various parameters to identify values resulting in treatment. For example, a subject may be made to sit in a chair or recline on a bed, after which muscle contractions (e.g., spasms) may be elicited by nerve electrical stimulation or the participant will trigger spasms naturally. In an example, ten pairs (i.e., 20 trials) of alternating unconditioned (no vibration) and conditioned (with vibration) trials will be recorded for each vibration frequency, duration, and amplitude. The EMG of the subject is collected to measure the effectivity of each set of parameters, i.e., to measure EMG signals in response to different vibratory stimulation signal treatments. The group vibration frequency, duration, and intensity that reduces EMG intensity and duration the most may then be stored in memory, for use by the process 208 upon subsequent muscle contraction conditions. If the results differ for spasm intensity and duration, that is, if different vibration parameters result in the greatest reduction in EMG intensity, while other vibration parameters result in the greatest reduction in EMG duration, then the system may store both different sets of vibration parameters, or the system may store the parameters that reduce EMG intensity the most since EMG intensity correlates strongly to self-reported spasm severity. For the vibration intensity parameter, the system would use the lowest vibratory stimulation amplitude that reduces muscle spasms. If different vibration parameters are needed to dampen spasms in a seated versus reclined posture.

In some examples, the EMG signals are sensed from the target muscle to be treated with the vibratory energy. In some examples, the EMG signals are sensed from an additional muscle other than the target muscle. In some examples, the vibratory energy is applied to the target muscle to treat the muscle contraction. In some examples, the vibratory energy is applied to the additional muscle to treat muscle contraction in the target muscle. In yet other examples, the vibratory energy (also termed a vibratory stimulus) is applied to a third target muscle different than the additional muscle and the target muscle to treat muscle contraction in the target muscle. Normally, joints are controlled by two opposite set of muscles: extensors and flexors. A vibration on the flexor muscle may also attenuate a spasm in the extensor one. Reciprocal inhibition may be the physiological mechanism involved in this spasm attenuation. Therefore, in some examples, the sensing EMG electrodes detect muscle contraction on the extensor, and the vibratory stimulus signals are applied by vibrating the flexor. Further, EMG electrodes may be placed on any number of target muscles on a subject, and the vibratory stimulus signals for treatment may be applied elsewhere. Because spinal circuits are responsible for spasm attenuation, a vibration on one muscle can affect the spinal circuits on many other muscles.

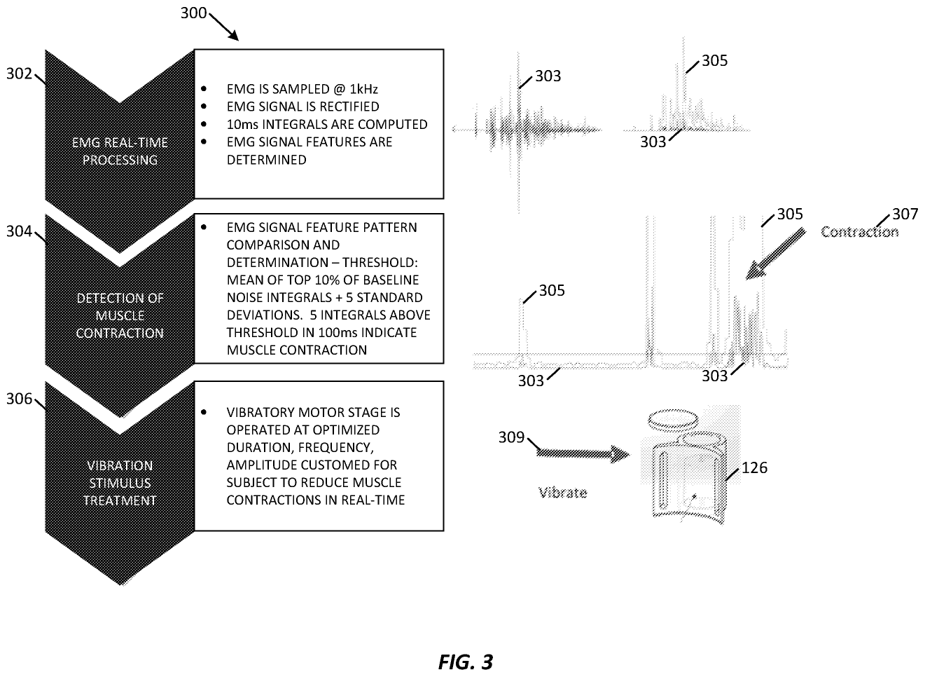

FIG. 3 illustrates a process 300 for detecting and treating muscle contractions in a subject. A detection algorithm is based on offline spasm classification rules, which have been previously developed and stored in a database, for example using testing of previous population samples and identifying EMG feature patterns indicating muscle contraction, e.g., spasm, etc. For example, the spasm classification rules may be developed from performing 24 hour recordings of muscle activity in a plurality of subjects.

At a process 302, EMG signal data is collected and, in the illustrated, sampled at 1 kHz. The EMG signals may be rectified (signal data 303) and integrated over 10 ms (signal data 305), in real-time. In the illustrated example, at a process 304, a threshold is calculated from the mean of the top 10% of baseline noise integrals plus 5 standard deviations. When 5 consecutive integrals are above threshold that is determined as indicating a muscle contraction (307). When muscle contraction is determined, a vibration motor is turned on for the optimal duration, frequency, and amplitude, as determined from by algorithms executed in a processor, at a process 306. The optimal direction, frequency, and amplitude of vibratory energy applied (309) to the subject is determined as those values that result in attenuation or complete removal of involuntary contractions in the target muscle. The end of a muscle contraction, e.g., the treatment thereof, may be indicated by 100 consecutive sampled and determined integrals being below threshold. After the muscle contraction has been treated, the process can turn off the vibratory motor and start sensing again for another contraction.

The present techniques provide numerous advantages over conventional devices. By having not just a portable device, but a wearable device, we are able to provide vibrational energy to treat the subject. Furthermore, instead of applying treatments at particular predetermined intervals, the present techniques can also apply treatment to specific muscle targets in real-time. Furthermore, the present techniques are able to attenuate involuntary muscle contractions. The present techniques are notable in that they are able to both detect and treat muscle contraction and of any detectable type, depending upon pre-determined classification rules. Furthermore, by operating in a closed loop manner, vibration treatment can be withheld when muscle contraction stops, which saves not only battery power but avoids situations where the subject adapts to the vibratory treatment and becomes desensitized to the treatment making it less effective. Further still the present techniques may be implemented as short term and long term treatment protocols, e.g., to treat short term muscle contractions as well to treat longer term muscle contraction problems.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components or multiple components. For example, references and illustrates herein to a “motion capture device,” motion capturing hardware device, and the like, include references to multiple ones of these devices as may be used attached to and/or separately spaced from a user. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a microcontroller, field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connects the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of the example methods described herein can be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method can be performed by one or more processors or processor-implemented hardware modules. The performance of certain of the operations may be distributed among the one or more processors, not only residing within a single machine, but also deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but also deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

This detailed description is to be construed as an example only and does not describe every possible embodiment, as describing every possible embodiment would be impractical, if not impossible. One could implement numerous alternate embodiments, using either current technology or technology developed after the filing date of this application.

What is claimed:

1. A system to treat muscle contraction of a subject, the system comprising:
one or more processors;
one or more memories;
a sensor stage having one or more electrodes configured to receive electromyography (EMG) signals from a target muscle of the subject;
a muscle contraction analysis stage configured to execute instructions stored on the one or more memories, wherein the instructions when executed cause the one or more processors to,
sample the EMG signals in real-time and calculate a plurality of features from the EMG signals,
determine whether a predetermined pattern of signal features indicate muscle contraction in the target muscle, and
if the pattern of signal features determine the presence of muscle contraction in the target muscle, determine the need for a vibratory stimulus signal to the muscle contraction; and
a vibratory motor stage configured to apply the vibratory stimulus signal to the subject to treat the muscle contraction;
wherein the sensor stage, the vibratory motor stage, and the muscle contraction analysis stage are in a closed-loop configuration, wherein the sensor stage is configured to receive additional EMG signals, the muscle contraction analysis stage is configured to sample the additional EMG signals in real-time and the vibratory motor stage is configured to stop application of the vibratory stimulus.

2. The system of claim 1, wherein the sensor stage is configured to sense the EMG signals at the target muscle.

3. The system of claim 2, wherein the vibratory motor stage is configured to apply the vibratory stimulus signal to the additional muscle as the target muscle.

4. The system of claim 2, wherein the vibratory motor stage is configured to apply the vibratory stimulus signal to the target muscle.

5. The system of claim 1, wherein the sensor stage is configured to sense the EMG signals in an additional muscle, other than the target muscle.

6. The system of claim 5, wherein the vibratory motor stage is configured to apply the vibratory stimulus signal to the additional muscle to treat muscle contraction in the target muscle.

7. The system of claim 5, wherein the vibratory motor stage is configured to apply the vibratory stimulus signal to a third muscle, different than the additional muscle and the target muscle to treat the muscle contraction in the target muscle.

8. The system of claim 1, wherein the sensor stage and the vibratory motor stage are housed in a bracelet having an engagement surface comprising the one or more electrodes and configured to engage a tendon attached to an additional muscle or to the target muscle, the bracelet having a vibratory motor housing mechanically coupled to the engagement surface, the vibratory motor housing configured to retain a vibratory motor for applying the vibratory stimulus signal generated by the vibratory motor to the engagement surface.

9. The system of claim 1, wherein the muscle contraction analysis stage comprises:
a power supply having a reference voltage, a DC to DC converter, and a low dropout voltage regulator.

10. The system of claim 9, wherein the muscle contraction analysis stage comprises:
an analog front end stage coupled to receive the EMG signals from the one or more electrodes;
an analog to digital converter; and
a vibratory motor microcontroller electrically coupled to the vibratory motor stage.

11. The system of claim 1, wherein the one or more electrodes comprises a first set of electrodes to be positioned at a first location on the target muscle and a second set of electrodes to be positioned at a second, different location on another target muscle.

12. A computer-implemented method to treat muscle contraction of a subject, the method comprising:
receiving, at a processor, electromyography (EMG) signals of the subject, the EMG signals being obtained from a target muscle of the subject;
sampling, at the processor, the EMG signals in real-time and calculating a plurality of features from the EMG signals;
determining whether a predetermined pattern of signal features indicate contraction in the target muscle;
if the pattern of signal features indicate the presence of muscle contraction in the target muscle and determining a vibratory stimulus signal corresponding to the muscle contraction and applying the vibratory stimulus signal to the subject to treat the muscle contraction; and
applying the vibratory stimulus signal in a closed-loop configuration, wherein the method further comprises receiving additional EMG signals and sampling the additional EMG signals in real-time, calculating a plurality of features of the additional EMG signal values, and determining when the plurality of feature patterns of the additional EMG signals indicate muscle contraction.

13. The method of claim 12, further comprising sensing the EMG signal of the subject at an additional muscle other than the target muscle.

14. The method of claim 13, further comprising applying the vibratory stimulus signal to the additional muscle to treat muscle contraction in the target muscle.

15. The method of claim 13, further comprising applying the vibratory stimulus to a third target muscle different than the additional muscle and the target muscle to treat muscle contraction in the target muscle.
